(19) United States
(12) Patent Application Publication
(10) Pub. No.: US 2008/0015458 A1
(43) Pub. Date: Jan. 17, 2008

(54) Methods of diagnosing and treating neuropsychological disorders

(76) Inventors: Pedro Steven Buarque de Macedo, Bethesda, MD (US); Ilene Sue Buarque de Macedo, Bethesda, MD (US)

Correspondence Address: AMSTER, ROTHSTEIN & EBENSTEIN LLP, 90 PARK AVENUE, NEW YORK, NY, 10016, US

(21) Appl. No.: 11/487,909

(22) Filed: Jul. 17, 2006

(51) Int. Cl. A61B 5/04

(52) U.S. Cl. 600/544

(57) ABSTRACT

Methods and apparatus are provided for diagnosing and/or treating neuropsychological disorders. The methods comprise: placing electrodes near superficial locations of peripheral nerves of a patient; detecting electro magnetic fields from said peripheral nerves of said patient; transmitting said detected electro magnetic fields to a wave form measuring device; and analyzing the detected electro magnetic fields as measured by said wave form measuring device. When said method is treating neuropsychological disorders, the method further comprises transmitting said detected electro magnetic fields to a wave form measuring device; analyzing the detected electro magnetic fields as measured by said wave form measuring device; and transmitting a mirrored electro magnetic field which is a function of the detected electro magnetic fields to said electrodes.

### FIELD OF THE INVENTION

The present invention relates generally to methods of, and apparatus for, diagnosing and/or treating neuropsychological disorders. More particularly, an embodiment of the present invention relates generally to a method of diagnosing neuropsychological disorders using electrodes located near superficial locations of peripheral nerves and analyzing the electromagnetic fields identified. Furthermore, an embodiment of the present invention relates generally to a method of treating neuropsychological disorders using electrodes located near superficial locations of peripheral nerves, analyzing the electromagnetic fields identified and transmitting modified electromagnetic fields at said superficial locations.

### BACKGROUND OF THE INVENTION

The brain is a vastly complex electromagnetic system. It is filled with neurons which are emitting electromagnetic radiation. The individual neurons have complex local ion fluxes and episodes of depolarization which cause them to emit an electromagnetic filed (EMF). The EMF has traditionally been evaluated from three views. The present invention relates to a new evaluation and treatment method.

A fundamental principle in electromagnetic theory is that a charge moving through a linear conduit will generate an EMF. Upon depolarization, the neuron 100, shown in FIG. 1 hereof, transmits an action potential down the axon 102 and generates a near field potential (NFP). These NFPs can be detected via percutaneous and surface electrodes 110 and are routinely measured in nerve conduction studies. This has been standard in neurological practice since the 1940s and is measured utilizing machinery and needles, which are well known in the art and commercially available.

The second method is the evaluation of Far Field Potentials (FFP). When a sensory system is stimulated such as vision, hearing, or touch, the neuronal pathways 122 in the brain 120, as shown in FIG. 2, carry an action potential along the axons 122, up through the spinal cord 124 and/or brainstem 126 into the cerebral hemispheres 128. This traveling action potential emits an EMF 130 which can be detected by surface or percutaneous antennae 132 on the scalp. This FFP is masked by the numerous other sources of EMF being emitted by the brain 120. However, by signal averaging hundreds to a few thousand stimulations of the sensory system, the random EMF is averaged out and the underlying FFP of the sensory stimulation is elucidated. This is called an evoked potential and has been a standard in neurological practice since the 1970s. It is measured utilizing machinery, surface electrodes and needles, which are well known in the art and commercially available.

The third method of evaluation of neuronal EMF is the Electroencephalogram (EEG). The gross EMF produced by the surface of the brain 120, as shown in FIG. 3 hereof, is measured on the scalp using surface or percutaneous electrodes 132. The EEG measures EMF 130 after it has passed through the skull. The skull acts as a high frequency filter of the EMF and dampens the amplitudes of the total EMF emitted. The EEG is analyzed in the 1 to 40 Hz frequency range. It represents the pacing or clocking of very large functional units within the brain 120. As with the other two methods it is a reflection of action potentials moving through axons near the cortical surface.

Unfortunately, none of these three prior art methods provides detailed information regarding the function of the brain at the dendritic level. Rather, the only information provided by these known techniques is information from the axon. What is needed is a method of analyzing information from the dendritic level regarding neuropsychological activities and a method of treating such neuropsychological activities based on the information analyzed. It is an object of the present invention to solve the shortcoming of the prior art.

It is a further object of the present invention to provide improved methods and apparatus for diagnosing and treating neuropsychological disorders, which involve using electrodes located near superficial locations of peripheral nerves of a patient to detect and analyze electro magnetic fields from the peripheral nerves.

### SUMMARY OF THE INVENTION

The present invention relates generally to methods of, and apparatus for, diagnosing and/or treating neuropsychological disorders. More particularly, an embodiment of the present invention relates generally to a method of diagnosing neuropsychological disorders using electrodes located near superficial locations of peripheral nerves and analyzing the electromagnetic fields identified. Furthermore, an embodiment of the present invention relates generally to a method of treating neuropsychological disorders using electrodes located near superficial locations of peripheral nerves, analyzing the electromagnetic fields identified and transmitting modified electromagnetic fields at said superficial locations.

A method is provided for diagnosing neuropsychological disorders comprising the following steps: placing electrodes located near superficial locations of peripheral nerves of a patient; detecting electro magnetic fields from said peripheral nerves of said patient; transmitting said detected electro magnetic fields to a wave form measuring device; and analyzing the detected electro magnetic fields as measured by said wave form measuring device. The peripheral nerves may comprise cranial nerves, including cranial nerve V (trigeminal), cranial nerve VII (facial), cranial nerve VIII (vestibulocochlear), cranial nerve IX (glossopharyngeal), cranial nerve X (vagal) and cranial nerve XI (spinal accessory), or peripheral nerves of the arms, peripheral nerves of legs, and paraspinal radicular nerves. The electrodes may be surface electrodes or percutaneous electrodes.

A method is also provided for treating neuropsychological disorders, which comprises the following steps: placing electrodes located near superficial locations of peripheral nerves of a patient; detecting electro magnetic fields from said peripheral nerves of said patient; transmitting said detected electro magnetic fields to a wave form measuring device; analyzing the detected electro magnetic fields as measured by said wave form measuring device; and transmitting a mirrored electro magnetic field which is a function of the detected electro magnetic fields to said electrodes. The peripheral nerves may comprise cranial nerves, including cranial nerve V (trigeminal), cranial nerve VII (facial), cranial nerve VIII (vestibulocochlear), cranial nerve IX (glossopharyngeal), cranial nerve X (vagal) and cranial nerve XI (spinal accessory), or peripheral nerves of the arms, peripheral nerves of legs, and paraspinal radicular nerves. The electrodes may be surface electrodes or percutaneous electrodes. In an embodiment of the invention the function of said mirrored electro magnetic field is represented as −αψ(θ+β)+γ, where α, β and γ are constants, and θ is the frequency of the measured wave form, and ψ(θ) is the detected electro magnetic field.

Apparatus is provided for diagnosing neuropsychological disorders comprising: electrodes for placement near superficial locations of peripheral nerves of a patient; amplifier and display for amplifying and displaying electro magnetic fields from said peripheral nerves of said patient; a wave form measuring device for measuring the waveform of said electro magnetic field; and a processing unit for analyzing said waveform of said electro magnetic field.

Apparatus is also provided for treating neuropsychological disorders, the apparatus comprising: electrodes for placement near superficial locations of peripheral nerves of a patient; amplifier and display for amplifying and displaying electro magnetic fields from said peripheral nerves of said patient; a wave form measuring device for measuring the waveform of said electro magnetic field; a processing unit for analyzing said waveform of said electro magnetic field and for generating a mirrored electro magnetic field which is a function of the detected electro magnetic field; and a device for transmitting said mirrored electro magnetic field to said electrodes.

These and other features of this invention are described in, or are apparent from, the following detailed description of various exemplary embodiments of the present invention.

### BRIEF DESCRIPTION OF THE DRAWINGS

Various exemplary embodiments of this invention will be described in detail, with reference to the following figures, wherein:

FIG. 1 is an illustration of a neuron and an electrode used to measure EMF waves of said neuron;

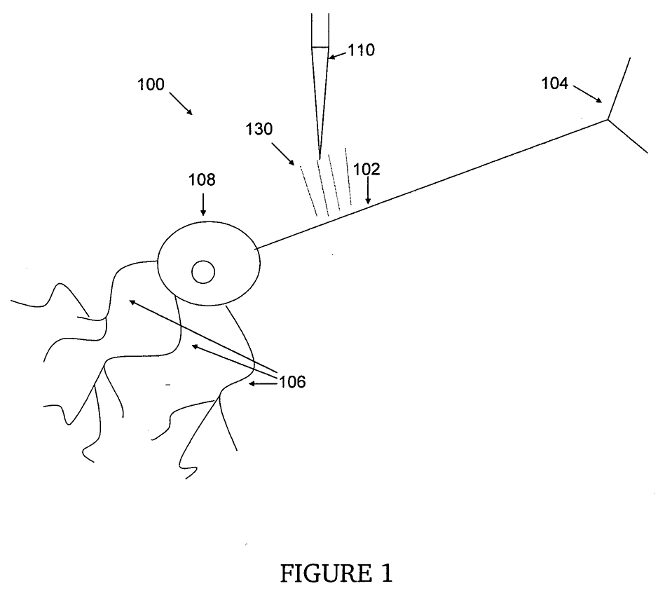

FIG. 2 is an illustration of a human brain being evaluated using FFP;

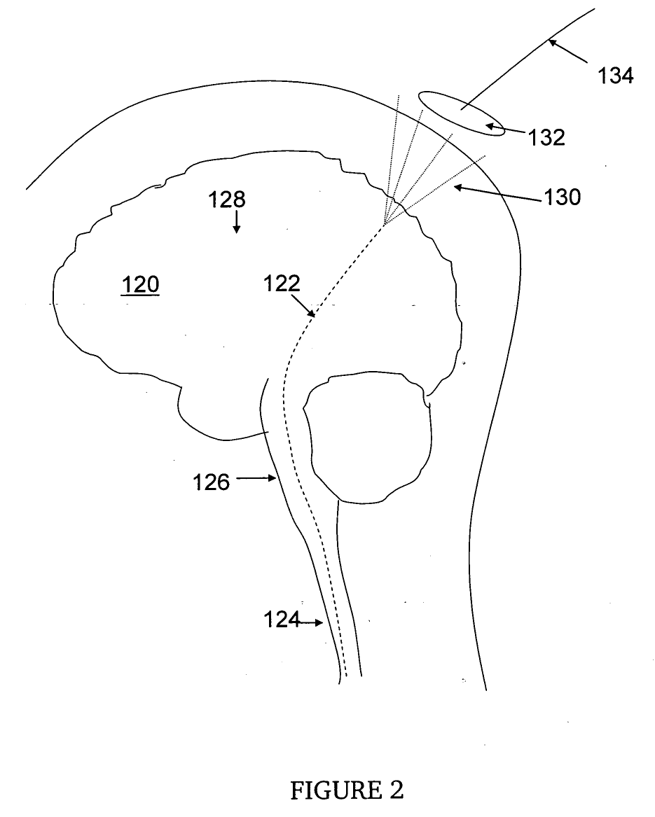

FIG. 3 is an illustration of a human brain being evaluated using EEG;

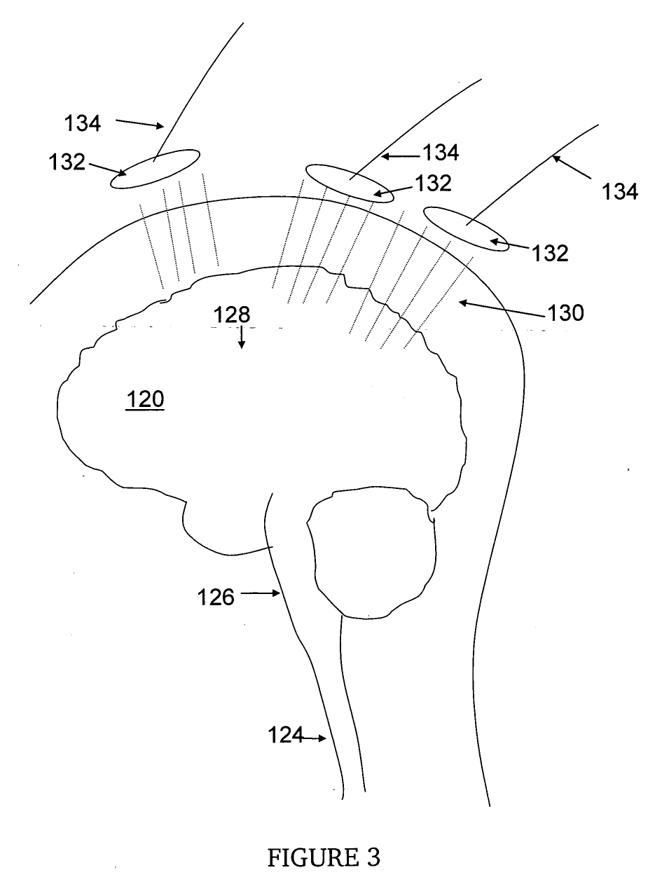

FIG. 4 is an illustration of a human brain being evaluated by an embodiment of the present invention;

FIG. 5 is an illustration of analysis equipment used with an embodiment of the present invention;

FIG. 6 is an illustration of analysis and treatment equipment used with an embodiment of the present invention.

### DETAILED DESCRIPTION OF PREFERRED EMBODIMENTS

The present invention relates to a new method of evaluation of neuronal EMF. All the previously known methods rely on analyzing action potentials as they transverse the neuron. The present invention instead detects the EMF produced by the local ion fluxes of the dendritic arborization. Each neuron has a unique location and dendritic pattern within the brain. Consequently the local ion fluxes of this dendritic region should be unique. Information analysis in the brain can be considered to occur at the dendrite level of the neuron. When there is sufficient depolarization at the dendrite, then an action potential will occur.

Different dendrite trees in the brain have different cerebral functions. For example one can postulate that depression is mediated by a specific cluster of dendrite trees. This cluster of dendrites has a unique pattern of ion fluxes and produces a unique EMF. This dendritic EMF has a much smaller amplitude than the axonal action potential EMF. Since ion fluxes occur at a much more rapid rate than action potentials, the dendritic EMF will also be higher frequency than the axonal action potential EMF, called “high frequency EEG” (hfEEG).

hfEEG would not be detectable on the surface of the scalp because the skull acts as a filter dampening the amplitude and frequencies of EEG as it transverses the skull. Consequently the hfEEG must be detected by antennae that are within the skull cavity (inside the skull). Subdural electrodes (those in which a wire is inserted surgically through the skull) are not practical for the vast majority of patients, consequently it is necessary to utilize naturally occurring anatomy as the subdural antennae.

There are a number of cranial nerves, which are functionally electric wires, which pass from the brain stem to the skull surface. These nerves include cranial nerves, such as cranial nerve V (trigeminal), cranial nerve VII (facial), cranial nerve VIII (vestibulocochlear), cranial nerve IX (glossopharyngeal), cranial nerve X (vagal) and cranial nerve XI (spinal accessory). The locations of peripheral nerves are well known to those of ordinary skill in the art. These nerves are essentially antennae which can be used to detect the hfEEG without any surgical intervention. Furthermore, peripheral nerves are in continuity with the spinal cord and the brain stem and also may function as antennae.

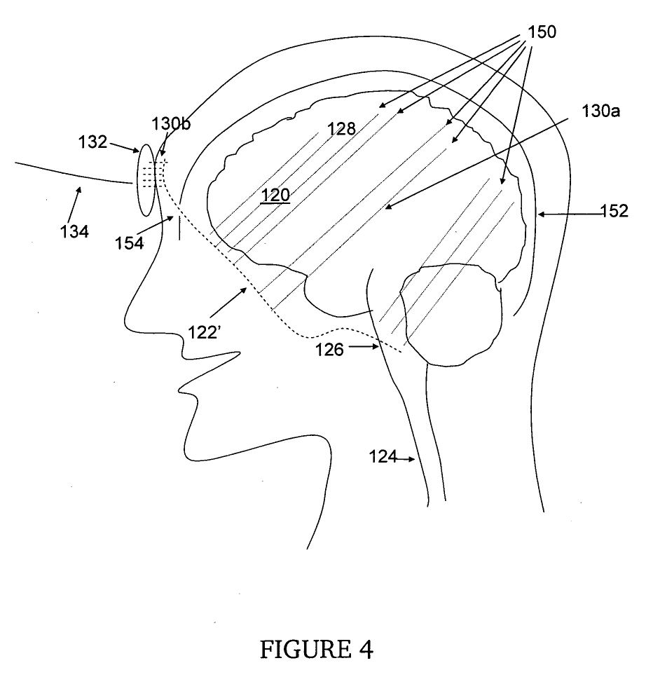

It is well established that the electric potential on the surface of nerves is very complex. In an embodiment of the present invention, as illustrated in FIG. 4 hereof, high frequency EMF 130a produced by the dendrite 150 of the cerebral cortex 128 and/or other parts of the brain 120 and being absorbed by the trigeminal nerve 122′ and being carried to the surface of the skull 152 along the trigeminal nerve 122′, which acts as an antennae, through exit opening 154. The high frequency EMF is then emitted, as high frequency EMF 130b, by the trigeminal nerve 122′ and detected by surface electrodes 132 or percutaneous electrodes. Both methods utilize the same equipment mentioned in the discussion of the near field potential and far field potential, as is well known in the art.

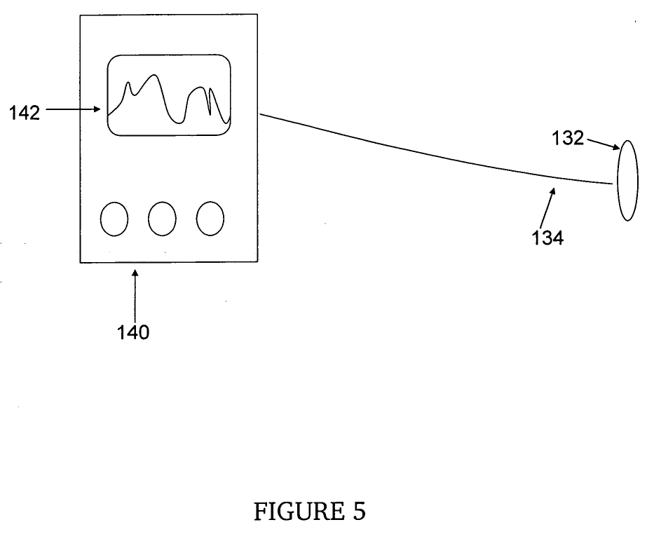

The signal detected by the surface electrode 132 or percutaneous electrodes is carried by wire 134 to a wave form measuring device 140, such as an oscilloscope, as shown in FIG. 5. The wave form ψ(θ) detected can then be correlated with particular brain functions such as attention, depression, vision, hearing, pain, etc. ψ(θ) is a waveform which is a function θ and can be displayed on the waveform measuring device 140. By analyzing the waveform ψ(θ) particular brain functions can be analyzed to diagnose neuropsychological disorders.

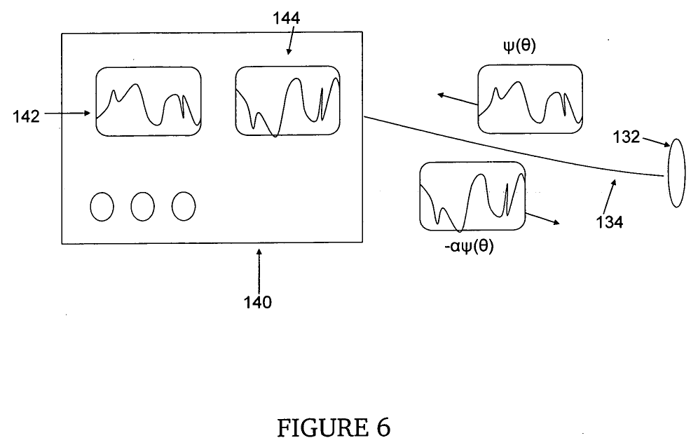

Once the particular wave form ψ(θ) associated with undesirable brain function is detected, in another embodiment of the invention, this undesirable brain function is treated as shown in FIG. 6, by transmitting a mirror wave form −αψ(θ) back along the wire 134 to the surface electrode 132 or percutaneous electrodes. The mirror wave form may be modulated in amplitude by a factor α. In another embodiment of the invention, the mirror wave form can also be modified in either phase or offset in a fixed amount, such that the mirror wave form can be represented as −αψ(θ+β)+γ, where α, β and γ are constants, and θ is the frequency of the measured wave form. In this embodiment of the present invention, a modulated mirror wave form which is a function of the detected wave form ψ(θ) is transmitted using surface electrode 132 or percutaneous electrodes to treat the undesirable brain function. In an embodiment of the present invention, β=0 and γ=0. In a further embodiment of the present invention, α is approximately 1, β is approximately 0 and γ is approximately 0.

While this invention has been described in conjunction with the exemplary embodiments outlined above, it is evident that many alternatives, modifications and variations will be apparent to those skilled in the art. Accordingly, the exemplary embodiments of the invention, as set forth above, are intended to be illustrative, not limiting. Various changes may be made without departing from the spirit and scope of the invention.

What is claimed is:

1. A method of diagnosing neuropsychological disorders comprising the following steps:
placing electrodes located near superficial locations of peripheral nerves of a patient;
detecting electro magnetic fields from said peripheral nerves of said patient;
transmitting said detected electro magnetic fields to a wave form measuring device; and
analyzing the detected electro magnetic fields as measured by said wave form measuring device.

2. The method of diagnosing neuropsychological disorders of claim 1, wherein said peripheral nerves comprise cranial nerves.

3. The method of diagnosing neuropsychological disorders of claim 2, wherein said cranial nerves comprise one or more of the following: cranial nerve V (trigeminal), cranial nerve VII (facial), cranial nerve VIII (vestibulocochlear), cranial nerve IX (glossopharyngeal), cranial nerve X (vagal) and cranial nerve XI (spinal accessory).

4. The method of diagnosing neuropsychological disorders of claim 1, wherein said peripheral nerves comprise one or more of the following: peripheral nerves of the arms, peripheral nerves of legs, and paraspinal radicular nerves.

5. The method of diagnosing neuropsychological disorders of claim 1, wherein said peripheral nerves comprise peripheral nerves of a leg.

6. The method of diagnosing neuropsychological disorders of claim 1, wherein said electrodes are surface electrodes.

7. The method of diagnosing neuropsychological disorders of claim 1, wherein said electrodes are percutaneous electrodes.

8. A method of treating neuropsychological disorders comprising the following steps:
placing electrodes located near superficial locations of peripheral nerves of a patient;
detecting electro magnetic fields from said peripheral nerves of said patient;
transmitting said detected electro magnetic fields to a wave form measuring device;
analyzing the detected electro magnetic fields as measured by said wave form measuring device; and
transmitting a mirrored electro magnetic field which is a function of the detected electro magnetic fields to said electrodes.

9. The method of treating neuropsychological disorders of claim 8, wherein said peripheral nerves comprise cranial nerves.

10. The method of treating neuropsychological disorders of claim 9, wherein said cranial nerves comprise one or more of the following: cranial nerve V (trigeminal), cranial nerve VII (facial), cranial nerve VIII (vestibulocochlear), cranial nerve IX (glossopharyngeal), cranial nerve X (vagal) and cranial nerve XI (spinal accessory).

11. The method of treating neuropsychological disorders of claim 8, wherein said peripheral nerves comprise one or more of the following: peripheral nerves of the arms, peripheral nerves of legs, and paraspinal radicular nerves.

12. The method of treating neuropsychological disorders of claim 8, wherein said peripheral nerves comprise peripheral nerves of a leg.

13. The method of treating neuropsychological disorders of claim 8, wherein said electrodes are surface electrodes.

14. The method of treating neuropsychological disorders of claim 8, wherein said electrodes are percutaneous electrodes.

15. The method of treating neuropsychological disorders of claim 8, wherein said function of said mirrored electro magnetic field is represented as −αψ(θ+β)+γ, where α, β and γ are constants, and θ is the frequency of the measured wave form, and ψ(θ) is the detected electro magnetic field.

16. The method of treating neuropsychological disorders of claim 15, wherein β=0 and γ=0.

17. The method of treating neuropsychological disorders of claim 16, wherein α is approximately 1, β is approximately 0 and γ is approximately 0.
